(19) United States
(12) Patent Application Publication
(10) Pub. No.: US 2021/0186074 A1
(43) Pub. Date: Jun. 24, 2021

(54) APPLICATIONS OF AN ESTER ADDITIVE FROM BIODERIVED RAW MATERIALS

(71) Applicant: FINE ORGANICS INDUSTRIES LTD., Mumbai (IN)

(72) Inventor: Mayukh Warawdekar, Navi Mumbai (IN)

(73) Assignee: FINE ORGANICS INDUSTRIES LTD., Mumbai (IN)

(21) Appl. No.: 16/769,410

(22) PCT Filed: Dec. 22, 2017
(86) PCT No.: PCT/IN2017/000143

§ 371 Date: Jun. 3, 2020

(51) Int. Cl. A23L 33/12; A23L 29/10; A61K 8/85; B65D 41/00

(52) U.S. Cl. CPC A23L 33/12; A23L 29/10; A61K 2800/87; B65D 41/005; A61K 2800/10; A61K 8/85

(57) ABSTRACT

An ester additive prepared from diol and dicarboxylic acid or fatty acid wherein the diol and dicarboxylic acid or fatty acid are bioderived and the ester is used as a slip and/or antiblocking agent and/or lubricant in moulded synthetic articles from polyvinylchloride, styrenics, thermoplastic elastomers, polyolefins and engineering plastics; in cosmetic compositions; in nutraceutical compositions and as food emulsifiers.

The present invention relates to an ester additive from bioderived diol and dicarboxylic acid or fatty acid for various applications.

### BACKGROUND OF THE INVENTION

The ester from bioderived resource refers to an ester comprising at least one repeating unit produced from diols and diacids/fatty acids derived from biomass resource such as fermentation of a carbon source by a microorganism.

Processes for preparing diacids such as succinic acid from biomass resources are disclosed in U.S. Pat. No. 8,203,021 (assigned to M/s. Bioamber S.A.S; referred to herein as the '021 patent).

Processes for preparing diols such as butanediol from biomass resources are also known from U.S. Pat. No. 8,410,291 (assigned to M/s. Bioamber International; referred to herein as the '291 patent)).

Japanese publication number 2006342227 (applicant M/s. Konica Minolta Opto Inc.; referred to herein as '227 publication) discloses manufacture of cellulose ester films for polarizers and liquid-crystal display devices with stable optical properties and good resistance to moisture. The film comprises polyesters prepared from glycol compounds having average carbon number of 2-3.5 and dibasic acids having average carbon number of 4-4.5.

PCT publication number 2013181580 by applicant M/s Bioamber Inc. (referred to herein as '580 publication) discloses composite panels or composite articles comprising at least one polyester resin adhesive and at least one lignin-based material and a method of preparing the composite panel or article.

PCT publication number 2014007988 by applicant M/s Teknor Apex Company (referred to herein as '988 publication) discloses novel carboxylic acid polyesters, and mixtures thereof, are useful as plasticizers such as for polyvinylchloride compositions. These polyesters and the other components of the mixtures are prepared from bio-renewable sources.

U.S. Pat. No. 8,912,233 (assigned to M/s. Surfatech Corporation; referred to herein as the '233 patent) discloses a series of polyesters synthesized utilizing propanediol with tunable ascetics and performance in cosmetic formulation.

U.S. Pat. No. 7,960,575 (assigned to M/s Dupont Tate & Lyle Bio products Company LLC.; referred to herein as the '575 patent) discloses process for the preparation of 1,3-propanediol distearate. A number of equivalent United States patents claim various applications of bio derived propylene glycol distearate such as personal care, detergents, flavours, agrochemicals and pharmaceutical excipients. However, there is no polymer application suggested in the '575 patent.

### OBJECT OF THE INVENTION

The object of the present invention is to provide an ester of 1,3 propanediol and succinic acid prepared from bioderived raw materials for use in polymers as slip and/or antiblocking agent and/or lubricant, cosmetic compositions, food emulsifiers, nutraceuticals and the like. The 1,3 propanediol and succinic acid ester additive of the present invention has good organoleptic properties.

Another object of the present invention is to provide a fatty ester additive prepared from bioderived raw materials for use in polymers as slip and/or antiblocking agent and/or lubricant, cosmetic compositions, food emulsifiers, nutraceuticals and the like. The fatty ester additive of the present invention has good organoleptic properties.

### SUMMARY OF THE INVENTION

An ester additive prepared from diol and dicarboxylic acid wherein the diol and dicarboxylic acid are bioderived and the ester is used as a slip and/or antiblocking agent and/or lubricant in moulded synthetic articles from polyvinylchloride, styrenics, thermoplastic elastomers, polyolefins and engineering plastics.

An ester additive prepared from diol and dicarboxylic acid wherein the diol and dicarboxylic acid are bioderived and the ester is used as a slip and/or antiblocking agent and/or lubricant in moulded synthetic articles from polyvinylchloride, styrenics, thermoplastic elastomers, polyolefins and engineering plastics; wherein the diol is 1,3-propanediol and the dicarboxylic acid is succinic acid.

A fatty ester additive prepared from diol and fatty acid wherein the diol and fatty acid are bio-derived and the ester is used as a slip and/or antiblocking agent and/or lubricant in moulded synthetic articles from polyvinylchloride, styrenics, thermoplastic elastomers, polyolefins and engineering plastics.

A fatty ester additive prepared from 1,3 propanediol and stearic acid wherein the 1,3 propanediol and stearic acid are bio-derived and the diester is used as a slip and/or antiblocking agent and/or lubricant in moulded synthetic articles from polyvinylchloride, styrenics, thermoplastic elastomers, polyolefins and engineering plastics.

### DESCRIPTION OF THE INVENTION

We have surprisingly found that an ester prepared from a diol and a dicarboxylic acid derived from biomass resource being environmentally friendly can be used in various applications such as a slip and/or antiblocking agent and/or lubricant in moulded synthetic articles from polyvinylchloride, styrenics, thermoplastic elastomers, polyolefins and engineering plastics; cosmetic compositions; food emulsifiers; nutraceuticals and the like. The diol and dicarboxylic acid or fatty acid being bioderived are easily sustainable or renewable as compared to ester prepared from synthetic diols and dicarboxylic acids or fatty acids. Also, the ester additive provides improved organoleptic properties to the articles as compared to existing slip and/or antiblocking agent and/or lubricants.

Similarly, a fatty ester prepared from a diol and fatty acid derived from biomass resource being environmentally friendly can be used in various applications such as a slip and/or antiblocking agent in polyolefins, cosmetic compositions, food emulsifiers, nutraceuticals and the like. Also, the fatty ester additive has good organoleptic properties.

According to one embodiment of the present invention is an ester prepared from diol and dicarboxylic acid which are bioderived. The ester may be used as a slip and/or antiblocking agent and/or lubricant in moulded synthetic articles from polyvinylchloride, styrenics, thermoplastic elastomers, polyolefins and engineering plastics, preferably polyolefins.

The diol may be selected from ethylene glycol, 1,3-propanediol, 1,4-butanediol, 1,5-pentanediol and 1,6-hexane diol.

The dicarboxylic acid may be selected from succinic acid, glutaric acid and adipic acid.

The polyolefin may be selected from polyethylene, polypropylene and ethylene vinyl acetate.

The moulded synthetic articles may be selected from caps, closures and the like. The cap or closure prepared by using the ester additive of the present invention may be used to seal liquid or food containers. The ester additive is stable at processing temperature range and retains color intensity of caps.

The cap using the ester of the present invention retains the organoleptic properties of the liquid as compared to erucamide. The liquids may be mineral water or carbonated drinks or non-carbonated drinks such as fruit juices.

The ester additive prepared from diol and dicarboxylic acid which are bioderived is used as a slip and/or antiblocking agent as it provides improved torque release characteristics to the moulded synthetic article such as polyolefin cap.

Additionally, the ester additive provides improved torque release characteristics to the polyolefin cap without generating any bad taste in the liquid or food stored in the container capped with the polyolefin caps.

The ester additive may also be used in cosmetic compositions, food emulsifiers, nutraceuticals and the like.

According to another embodiment of the present invention is a an ester prepared from 1,3 propanediol and succinic acid which are bioderived. The ester may be used as a slip and/or antiblocking agent and/or lubricant in moulded synthetic articles prepared from polyvinylchloride, styrenics, thermoplastic elastomers, polyolefins and engineering plastics, preferably polyolefins.

The moulded synthetic articles may be selected from caps, closures and the like. The cap or closure prepared by using the polypropylene cap composition of the present invention may be used to seal liquid or food containers. The ester additive of 1,3 propanediol and succinic acid is stable at processing temperature range and retains color intensity of caps.

The cap using the ester additive prepared from 1,3 propanediol and succinic acid of the present invention retains the organoleptic properties of the liquid as compared to erucamide. The liquids may be mineral water or carbonated drinks or non-carbonated drinks such as fruit juices.

The 1,3 propane diol succinate ester additive prepared from 1,3 propanediol and succinic acid which are bioderived is used as a slip and/or antiblocking agent as it provides improved torque release characteristics to the moulded synthetic article such as polyolefin cap.

Additionally, the 1,3 propane diol disuccinate ester additive provides improved torque release characteristics to the polyolefin cap without generating any bad taste in the material stored in the bottle capped with the polyolefin caps.

The 1,3 propane diol succinate diester additive may also be used in cosmetic compositions, food emulsifiers, nutraceuticals and the like.

According to yet another embodiment of the present invention is a fatty ester comprising at least one repeating unit prepared from 1,3 propanediol and a fatty acid selected from C₁₂ to C₂₂ acid which are derived from edible vegetable oils. The fatty ester may be used as a slip and/or antiblocking agent and/or lubricant in moulded synthetic articles prepared from polyvinylchloride, styrenics, thermoplastic elastomers, polyolefins and engineering plastics, preferably polyolefins.

The moulded synthetic articles may be selected from caps, closures and the like. The cap or closure prepared by using the polypropylene cap composition of the present invention may be used to seal liquid or food containers. The fatty ester additive of 1,3 propanediol and fatty acid is stable at processing temperature range and retains color intensity of caps.

The cap using the fatty ester of the present invention retains the organoleptic properties of the liquid as compared to erucamide. The liquids may be mineral water or carbonated drinks or non-carbonated drinks such as fruit juices.

The fatty ester additive is used as a slip and/or antiblocking agent as it provides improved torque release characteristics to the moulded synthetic article such as polyolefin cap.

Additionally, the fatty ester additive provides improved torque release characteristics to the polyolefin cap without generating any bad taste in the material stored in the bottle capped with the polyolefin caps.

The fatty ester of 1,3 propanediol and fatty acid may also be used in cosmetic compositions, food emulsifiers, nutraceuticals and the like.

The preferred embodiment of the present invention is a fatty ester such as 1,3 propanediol distearate comprising at least one repeating unit prepared from 1,3 propanediol and stearic acid which are derived from edible vegetable oils. The 1,3 propanediol distearate may be used as a slip and/or antiblocking agent and/or lubricant in moulded synthetic articles prepared from polyvinylchloride, styrenics, thermoplastic elastomers, polyolefins and engineering plastics, preferably polyolefins.

The moulded synthetic articles may be selected from caps, closures and the like. The cap or closure prepared by using the polypropylene cap composition of the present invention may be used to seal liquid or food containers. The 1,3 propanediol distearate is stable at processing temperature range and retains color intensity of caps.

The cap using the 1,3 propanediol distearate of the present invention retains the organoleptic properties of the liquid as compared to erucamide. The liquids may be mineral water or carbonated drinks or non-carbonated drinks such as fruit juices.

The 1,3 propanediol distearate is used as a slip and/or antiblocking agent as it provides improved torque release characteristics to the moulded synthetic article such as polyolefin cap.

Additionally, the 1,3 propanediol distearate provides improved torque release characteristics to the polyolefin cap without generating any bad taste in the material stored in the bottle capped with the polyolefin caps.

The 1,3 propanediol distearate may also be used in cosmetic compositions, food emulsifiers, nutraceuticals and the like.

### DEFINITION OF TERMS

The term torque release is the moment of a force tending to cause rotation of an appropriate closure in a direction opposite to that of closing, causing the closure to be unsecured from its position on the neck finish of an appropriate container.

The term Organoleptic Property is the result of adverse effect of substance/material on smell and taste. It can be assessed by human panel measured as organolepticity index or by using sophisticated analytical tools.

The following examples illustrate preferred embodiments in accordance with the present invention without limiting the scope of the invention.

### EXAMPLES

### Example 1

Test Method for the Determination of Torque Release of the Caps

Summary: The representative injection moulded caps are held in torque meter, the specific closing torque is applied and then the removal torque (the torque necessary to loosen the closure) is determined.

Apparatus:

1. Torque Meter: The scale 0 to 6.0 Nm, minimum accuracy 0.1 Nm (See Picture)

2. The Container Holder: To hold the container firmly on its place, so that the measurement can be taken accurately.

3. The Closure Fixture: Of the appropriate size of the closure (for the mineral water and carbonated cold drink containers)

Conditioning:

Condition the closures at room temperature for minimum 48 hrs (2 days) before the testing.

Procedure:

1. Firmly position the container (mineral water bottle or carbonated cold drink bottle) in the container holder. Fix the closure or the cap under test in the fixture.

2. Fix the fixture (with the closure inside) to the container, avoiding any contact with the container.

3. Fix the torque meter, keeping red pointer at left side. Rotate clockwise slowly, at constant rate, watching the gauge until the desired/predetermined constant torque is indicated on the needle.

4. Keeping the torque meter in its place, bring the red needle to the right hand side, rotate anticlockwise, slowly, watching the gage, avoiding any contact with the container. The highest figure indicated will be release or removal torque of the closure.

5. Minimum 10 caps/closures should be tested after 48 hrs conditioning and thereafter 5, 10, 15 and 30 days OR till the constant release torque is obtained.

6. Measure dimensions and weights of the caps under test. (10 nos. minimum)

Report:

a. Closing Torque, Release Torque (Avg. of 10 values), in Nm, after 2, 5, 10, 15 and 30 days.

b. Polymer used for the closures.

c. Additive concentration.

### Example 2

### % Torque Release of HDPE Caps—Mineral Water Bottle

Polymer: HDPE (MB5568) of Borealis, MFI 0.8 g/10 min @190° C., 2.16 kg

Process: Master batch preparation (5% w/w) followed by injection molding.

Conditioning: 48 hrs at R.T. to release stresses developed during molding.

Testing: Release torque measurement by keeping the closing torque constant.

Closing torque: 3.0 Nm
A. 2500 ppm of additive

| | Torque Release Nm after no of days | | | | |
|---|---|---|---|---|---|
| Sample | 2 | 5 | 10 | 15 | 30 |
| HDPE control | 0.97 | 0.86 | 0.92 | 0.88 | 0.87 |
| HDPE + behenamide | 0.48 | 0.42 | 0.45 | 0.42 | 0.44 |
| HDPE + isostearamide | 0.82 | 0.69 | 0.70 | 0.58 | 0.60 |
| HDPE + 1,3 propanediol | 0.82 | 0.67 | 0.70 | 0.61 | 0.64 |
| disuccinate | | | | | |

A. 5000 ppm of additive

| | Torque Release Nm after no of days | | | | |
|---|---|---|---|---|---|
| Sample | 2 | 5 | 10 | 15 | 30 |
| HDPE control | 0.97 | 0.85 | 0.86 | 0.86 | 0.87 |
| HDPE + behenamide | 0.48 | 0.45 | 0.43 | 0.40 | 0.40 |
| HDPE + isostearamide | 0.62 | 0.58 | 0.55 | 0.52 | 0.48 |
| HDPE + 1,3 propanediol | 0.75 | 0.62 | 0.57 | 0.58 | 0.55 |
| disuccinate | | | | | |

### Example 3

### Color Heat Stability Data @205° C., 1 hr

GARDNER COLOR

| SAMPLE | Initial | 205° C. 1 hr |
|---|---|---|
| HDPE + 1,3 propanediol disuccinate | 0.6 | 0.6 |
| HDPE + isostearamide | 0.6 | 2.8 |
| HDPE + 1,3 propanediol distearate | 0.4 | 0.4 |

### Example 4

### Torque Release

Polymer: HDPE (MB5568) of Borealis, MFI 0.8 g/10 min @190° C., 2.16 kg.

Applications: HDPE Caps & Closures

Process: Masterbatch preparation (5%, w/w) followed by Injection molding.

Conditioning: 48 hrs at Room temperature to release stresses developed during molding.

Testing: Release torque measurement by keeping the closing torque constant.

Caps: Mineral water bottle caps

Results:

| | Closing Torque 3.0 Nm | | | | |
|---|---|---|---|---|---|
| | Torque Release Nm | | | | |
| | 2,500 ppm | | | | |
| | Days | | | | |
| Sample | 2 | 5 | 10 | 15 | 30 |
| HDPE Control | 1.10 | 0.98 | 1.00 | 1.00 | 1.02 |
| HDPE + Behenamide | 0.70 | 0.67 | 0.66 | 0.61 | 0.67 |
| HDPE + Erucamide | 0.40 | 0.36 | 0.27 | 0.23 | 0.23 |
| HDPE + behenamide:glycerolmonostearate (25:75) | 0.75 | 0.67 | 0.68 | 0.65 | 0.67 |
| HDPE + Lauramide | 0.70 | 0.65 | 0.66 | 0.64 | 0.66 |
| HDPE + 1,3 propanediol disuccinate | 0.80 | 0.68 | 0.65 | 0.62 | 0.60 |
| HDPE + 1,3 propanediol distearate | 0.66 | 0.63 | 0.63 | 0.64 | 0.62 |

### Example 5

### Organolepticity Index

(a) Test Method for the Determination of Organolepticity for the Caps & Closures After Exposure to Sun

This method determines the Organolepticity Index of caps & closures, fitted on the glass bottles filled with potable water, after exposure to sunlight.

Summary:

The representative injection moulded closures were fitted on glass bottles containing potable water and are exposed to sunlight for 72 hours. A panel of 10-12 employees tested the water for taste and odour and allotted a rank ranging from 1-4. Higher the rank poorer the taste.

Apparatus:

Glass bottles, tray, table, glasses, hand gloves, etc.

Conditioning:

Condition the closures at room temperature for minimum 48 hrs (2 days) before the testing.

Procedure:
1. 500 ml Glass bottles were filled with potable water & fitted with test caps and a control (Cap made without additive).
2. These bottles were laid horizontally in a tray and exposed to direct sunlight for 72 hrs.
3. Bottles were shaken occasionally [thrice a day] to maintain the homogeneous contact of water with the cap.
4. After 72 hrs, of exposure to sunlight, the bottles were cooled to ambient temperature.
5. Panelists (10-12 members) were invited to taste the water and smell the contents
6. Each panellist ranked the caps from 1 to 4; a high mark corresponds to bad taste.
7. The Organolepticity index (OI) was the mean of the marks given by the panelist.

Report:
Report the following information
Type of the caps.
Polymer of the cap
Additive dosage level
Organolepticity Index (OI).
   (b) Rating based on 10 no panelist report is as follows

| ADDITIVE | |
|---|---|
| Control | RATING# |
| Behenamide | 1.8 |
| Erucamide | 3.7 |
| behenamide:glycerolmonostearate (25:75) | 2.3 |
| Lauramide | To be done |
| 1,3 propanediol disuccinate | 2.4 |
| 1,3 propanediol distearate | To be done |

#higher the value, worse the taste on a scale of 1 to 4

1-11. (canceled)

12. An ester additive prepared from diol and dicarboxylic acid wherein the diol and dicarboxylic acid are bioderived and the ester is used as a slip and/or antiblocking agent and/or lubricant in moulded synthetic articles from polyvinylchloride, styrenics, thermoplastic elastomers, polyolefins and engineering plastics; wherein the diol is 1,3-propanediol and the dicarboxylic acid is succinic acid.

13. An ester as claimed in claim 12 wherein the polyolefin is selected from polyethylene, polypropylene and ethylene vinyl acetate.

14. An ester as claimed in claims 12 and 13 wherein the moulded synthetic article from polyolefins is cap or closure.

15. An ester as claimed in claim 14 wherein the cap or closure is used to seal liquid or food containers.

16. An ester as claimed in claim 15, wherein the cap enables retention of the organoleptic properties of the liquid selected from mineral water, carbonated drinks and non-carbonated drinks such as fruit juices.

17. An ester additive comprising at least one repeating unit prepared from a 1,3-propanediol and succinic acid wherein the 1,3-propanediol and succinic acid are bioderived and the ester is used in nutraceutical composition or cosmetic composition or as food emulsifier.

18. An ester additive as claimed in claim 12 wherein the ester additive provides improved torque release characteristics to the moulded synthetic article.

19-29. (canceled)

30. A fatty ester additive prepared from 1,3 propanediol and stearic acid wherein the 1,3 propanediol and stearic acid are bioderived and the diester is used as a slip and/or antiblocking agent and/or lubricant in moulded synthetic articles from polyvinylchloride, styrenics, thermoplastic elastomers, polyolefins and engineering plastics.

31. A fatty ester as claimed in claim 30 wherein the polyolefin is selected from polyethylene, polypropylene and ethylene vinyl acetate.

32. A fatty ester as claimed in claims 30 and 31 wherein the moulded synthetic article from polyolefins is cap or closure.

33. A fatty ester as claimed in claim 30 wherein the cap or closure is used to seal liquid or food containers.

34. A fatty ester as claimed in claim 30, wherein the cap enables retention of the organoleptic properties of the liquid selected from mineral water, carbonated drinks and non-carbonated drinks such as fruit juices.

35. A fatty ester additive comprising at least one repeating unit prepared from 1,3 propanediol and stearic acid wherein the diol and dicarboxylic acid are bio-derived and the fatty ester is used in nutraceutical composition or cosmetic composition or as food emulsifier.

36. A fatty ester additive as claimed in claim 30 wherein the fatty ester additive provides improved torque release characteristics to the moulded synthetic article.
